(19) United States
(12) Patent Application Publication
(10) Pub. No.: US 2003/0144220 A1
(43) Pub. Date: Jul. 31, 2003

(54) Use of CYP2D6 inhibitors in combination therapies

(76) Inventor: R. Scott Obach, Gales Ferry, CT (US)

Correspondence Address: PFIZER INC, 150 EAST 42ND STREET, 5TH FLOOR - STOP 49, NEW YORK, NY, 10017-5612, US

(21) Appl. No.: 09/528,978

(22) Filed: Mar. 21, 2000

Related U.S. Application Data
(60) Provisional application No. 60/128,136, filed on Apr. 7, 1999.

(51) Int. Cl. A61K 31/7056; A61K 31/4745; A61K 31/542; A61K 31/137; A61K 31/498

(52) U.S. Cl. 514/43; 514/249; 514/283; 514/305; 514/603; 514/317; 514/225.2; 514/650

(57) ABSTRACT

This invention relates to the use of a CYP2D6 inhibitor in combination with a drug having CYP2D6 catalyzed metabolism, wherein the drug and the CYP2D6 inhibitor are not the same compound; and pharmaceutical compositions for said use.

### BACKGROUND

This invention relates to the use of a CYP2D6 inhibitor in combination with a drug having CYP2D6 catalyzed metabolism in order to improve the drug's pharmacokinetic profile.

The clearance of drugs in humans can occur by several mechanisms, such as metabolism, excretion in urine, excretion in bile, etc. Despite the many types of clearance mechanisms, a large proportion of drugs are eliminated in humans via hepatic metabolism. Hepatic metabolism can consist of oxidative (e.g., hydroxylation, heteroatom dealkylation) and conjugative (e.g., glucuronidation, acetylation) reactions. Again, despite the many possibilities of types of metabolic reactions, a preponderance of drugs are metabolized via oxidative pathways. Thus, the primary route of clearance of a vast majority of drugs is oxidative hepatic metabolism.

Of the enzymes involved in the oxidative metabolism of drugs, the cytochrome P-450 (CYP) superfamily of enzymes are major contributors. CYP constitutes a class of over 200 enzymes that are able to catalyze a variety of types of oxidative reactions (via a hypothesized common reaction mechanism) on a wide range of xenobiotic substrate structures. In humans, the CYP catalyzed metabolism of most drugs is carried out by one of five isoforms: CYP1A2, CYP2C19, CYP2C9, CYP2D6, and CYP3A4, with the latter three being the most important of these enzymes.

Of all of the known human CYP isoforms, the most highly developed knowledge base of substrate specificity is for CYP2D6. This isoform is almost exclusively involved in the oxidative metabolism of lipophilic amine drugs. Well known CYP2D6 substrates include neuroleptics, type 1C antiarrhythmics, β-blockers, antidepressants (tricyclic antidepressants, selective serotonin reuptake inhibitors and monoamine oxidase inhibitors), and others such as codeine and dextromethorphan. This apparent specificity for amines as substrates is hypothesized to arise from the presence of an acidic amino acid residue in the substrate binding site. This residue can form an ionic interaction with amine substrates while positioning sites for oxidation in propinquity to the reactive iron center of the heme of CYP. Structure activity relationships for CYP2D6 and the metabolism of amines have led to the development of a predictive model for this enzyme which states that the position of oxidation of a CYP2D6 substrate is 5 to 7 Å from the basic amine nitrogen. Some additional steric requirements are also hypothesized.

Many compounds for which the major clearance mechanism in humans is CYP2D6 mediated oxidative biotransformation commonly exhibit one or more detrimental characteristics with regard to human pharmacokinetics. These characteristics are: (1) wide disparity in exposure between individuals possessing and lacking a copy of the CYP2D6 gene (“extensive and poor metabolizers”); (2) high inter-individual variability in exposure among extensive metabolizers; (3) propensity for supraproportional dose-exposure relationships; (4) frequent drug-drug interactions; and (5) short half-lives and poor oral bioavailability due to extensive first-pass hepatic clearance.

While not all CYP2D6 substrates possess these characteristics, most CYP2D6 substrates are subject to one or more.

In the mid-1980s observations were made concerning the disparity in exposure to drugs in a small subset of the population. In some cases, the high exposures observed in the minority of individuals were also associated with adverse reactions. These observations led to the discovery of the CYP2D6 genetic polymorphism. The CYP2D6 gene is absent in 5-10% of the Caucasian population (referred to as poor metabolizers or PM's). Such individuals can be distinguished from the rest of the population (extensive metabolizers or EM's) by an examination of genotype through restriction fragment length polymorphism analysis or through determination of phenotype by measurement of the urinary dextrorphan/dextromethorphan ratio after administration of the latter compound. When population histograms of exposure to prototypical CYP2D6-cleared compounds are constructed, a bimodal distribution is observed. For example, the mean terminal phase half-life of propafenone, a well known CYP2D6 cleared compound, is 5.5 hours in extensive metabolizers, but is 17.2 hours in poor metabolizers. EM-PM differences are typically exacerbated upon oral administration of CYP2D6 cleared compounds due to wide disparities in first-pass extraction. Propafenone exposure after oral administration is 4.2-fold greater in PM's vs. EM's. Thus, CYP2D6 cleared compounds can be subject to increased incidences of adverse effects, due to elevated systemic exposures observed in PM's.

Regardless of the genetic polymorphism, a high degree of interindividual variability exists in the exposure to CYP2D6 cleared compounds among those individuals considered to be extensive metabolizers. While a reason for this variability is not presently known, it does not appear to be due to an increase in CYP2D6 gene copy number (although one such genotype has been reported in the literature in Sweden), nor does it appear to be due to environmental factors as this CYP isoform has never been demonstrated to be inducible. An example of this variability phenomenon is demonstrated by the exposure to the antidepressant agent imipramine and its metabolite desipramine, which demonstrates a 20-fold range of steady state plasma concentrations after oral administration. For compounds with wide therapeutic indices, this variability may not be problematic. However, if the therapeutic index for a CYP2D6 cleared compound approaches 10, increased incidences of adverse effects are likely to be observed.

Metabolic clearance is a potentially saturable process. The intrinsic clearance (Cl′ᵢₙₜ, the ability of an organ to clear a compound without constraints imposed by organ blood flow or plasma protein binding) is a function of Michaelis-Menten parameters:

$$\frac{1}{oral exposure} ∝ {Cl}_{int}^{′} = \frac{{V}_{max}}{{K}_{M} + [ S ]}$$

where both Vₘₐₓ and K_{M} are fixed constants and [S] represents the concentration of the drug in the clearing organ. For most drugs, concentrations of drug typically attained in vivo are well below the K_{M} and thus the denominator of the above expression degenerates to a constant value of K_{M}. However, for many CYP2D6 catalyzed reactions, K_{M} values are typically low. This is hypothesized to be due to the strong (relative to other CYP enzymes) ionic bond formation between cationic amine substrates and an anionic amino acid in the substrate binding site of CYP2D6. Thus for compounds cleared by CYP2D6, drug concentrations can approach and exceed K_{M} values resulting in intrinsic clearance values that decrease with increasing drug concentration. Since drug concentration is related to dose, clearance is observed to decrease with increasing dose. With decreases in clearance with increases in dose, exposure is thus observed to increase in a supraproportional manner with increasing dose. Such a relationship has been described in the scientific literature for the CYP2D6 cleared compounds propafenone and paroxetine. Interestingly, this phenomenon is not observed in poor metabolizers, since the CYP2D6 isoform is not present in these individuals.

The parameter K_{M} is a complex function of enzymatic rate constants that, for CYP, has a strong component of substrate binding rate constants. The potential exists that competitive inhibition of the metabolism of one drug can occur via catalytically competent substrate binding of a second drug. Since the K_{M} for CYP enzymes are closely related to binding constants, they approximate Kᵢ values in many cases. For CYP2D6, low K_{M} values for typical substrates can also result in low Kᵢ values for these same substrates as competitive inhibitors. Low Kᵢ values reflect a greater potential to result in drug-drug interactions, since lower concentrations and doses of drug are adequate to exhibit inhibition. Thus, the potential for drug-drug interactions is a more likely concern with CYP2D6 substrates than other CYP substrates, due to the greater binding affinities of the former. Thus, since Kᵢ values typically track K_{M} values, the potential for drug-drug interactions usually go hand-in-hand with the potential for supraproportional dose-exposure relationships.

As mentioned above, clearance is related to the term Vₘₐₓ/K_{M}. For compounds with similar Vₘₐₓ values, the lower the value for K_{M}, the higher the clearance. Since many CYP2D6 substrates have very low K_{M} values, these compounds, as a class, are more likely to exhibit high hepatic clearance in vivo. High hepatic clearance results in shorter half-lives. It also results in greater first-pass hepatic extraction which can result in low oral bioavailabilities. This point is represented by the compounds (7S,9S)-2-(2-pyrimidyl)-7-(succinamidomethyl)-prehydro-1H-pyrido-[1,2-a]pyrazine) (“sunipetron”) (K_{M} of about 1 μM, human half-life of about 1 hour), (2S,3S)-2-phenyl-3-(2-methoxyphenyl)-methylaminopiperidine (K_{M} of about 1 μM, human half-life of about 4.7 hours), (1S,2S)-1-(4-hydroxyphenyl)-2-(4-hydroxy-4-phenylpiperidin-1-yl)-1-propanol (K_{M} of about 3-4 μM, human half-life of about 3-4 hours), and (2S,3S)-2-phenyl-3-(2-methoxy-5-trifluoromethoxyphenyl)-methylamino-piperidine (K_{M} of about: 1 μM, human half-life of about 8 hours), all of which are CYP2D6 substrates. The former two compounds have K_{M} values in the 1 μM range. The human half-lives for these two compounds are 1.1 and 4.7 hours, and human oral bioavailability values for these two compounds are 4.6 and 1.0%, respectively. The clearance values for the former two compounds, measured after intravenous administration to humans, are in the range of blood-flow limiting values, suggesting that hepatic extraction exceeds 90%.

There are several compounds known to inhibit CYP2D6 reactions, either by ‘pure’ inhibition or by acting as competitive substrates. Unlike many other CYP enzymes, there are some potent inhibitors known for CYP2D6. Again, it is believed that the ionic interaction between the cationic amine group of the inhibitor and the anionic amino acid residue of CYP2D6 is at least partially responsible for the potency of CYP2D6 inhibitors. Two examples of potent CYP2D6 inhibitors are quinidine and ajmalacine:

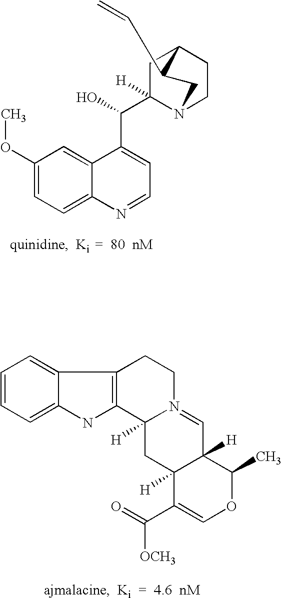

Quinidine represents a commonly utilized antiarrhythmic agent whereas ajmalacine is a less well-known natural product with vasodilation activity. Since quinidine is a commonly administered substance, drug interaction studies have been conducted in vivo for this drug and CYP2D6 cleared compounds. Quinidine has the effect of converting an extensive metabolizer to the poor metabolizer phenotype via inhibition of CYP2D6.

In addition, extracts of St. John's wort have recently been found to contain constituent substances that exhibit CYP inhibitory activity, including inhibition of CYP2D6. Examples of constituent substances of St. John's extract that exhibit CYP inhibitory activity are hyperforin, 13, 118-biapigenin, hypericin, and quercetin. Other unidentified components also exhibit CYP inhibitory activity.

For CYP2D6 cleared compounds, the problem that is frequently focused on is the disparity in the exposures between extensive and poor metabolizers and the high variability demonstrated by the extensive metabolizers. However, what is commonly overlooked is the fact that these compounds typically have very satisfactory pharmacokinetics in the poor metabolizers. In subjects lacking the CYP2D6 enzyme, CYP2D6 cleared compounds: (1) typically have long t_{1/2} values and high oral bioavailability and (2) do not exhibit supraproportional dose-exposure relationships. By lacking the CYP2D6 enzyme, the variability of drug exposures in poor metabolizers is no greater than variabilities exhibited by non-CYP2D6 cleared compounds. Although attempts have been made to link poor metabolizer status with proclivity to various pathological states, a definitive cause-effect relationship has yet to be established. Thus, since poor metabolizers represent a normal and healthy segment of the population, it is not anticipated that converting extensive metabolizers to poor metabolizers via administration of a specific CYP2D6 inhibitor would result in any untoward effects related to inhibition of this enzyme.

This invention relates to the coformulation or combined use of a CYP2D6 inhibitor and a CYP2D6 cleared compound. Thus, instead of avoiding a drug-drug interaction, this invention involves developing such an interaction intentionally in order to improve the pharmacokinetics of therapeutically useful, but pharmacokinetically flawed compounds. Such an approach is analogous to the utilization of sustained-release formulations to enhance the pharmacokinetics of drugs. However, instead of modulating drug elimination via input rate limitation, this approach seeks to do the same by modulating the elimination rate directly. Furthermore, in addition to lengthening half-life, a CYP2D6 inhibitor would enhance oral exposure due to a suppression of hepatic first-pass extraction.

### SUMMARY OF THE INVENTION

This invention relates to a method of administering a drug for which the major clearance mechanism in humans is CYP2D6 mediated oxidative biotransformation (also referred to throughout this document as a “Therapeutic Drug”), or a pharmaceutically acceptable salt thereof, in combination with a CYP2D6 inhibitor, or a pharmaceutically acceptable salt thereof, to a human in need of the intended pharmaceutical activity of such drug, wherein the Therapeutic Drug and the CYP2D6 inhibitor are not the same compound. The above method is hereinafter referred to as the “Combination Method”.

This invention also relates to the Combination Method, wherein the drug for which the major clearance mechanism in humans is CYP2D6 mediated oxidative biotransformation is a selective serotonin reuptake inhibitor containing a primary, secondary or tertiary alkylamine moiety (e.g., sertraline or fluoxetine).

This invention also relates to the Combination Method, wherein the drug for which the major clearance mechanism in humans is CYP2D6 mediated oxidative biotransformation is an NMDA (N-methyl-D-aspartate) receptor antagonist containing a primary, secondary or tertiary alkylamine moiety.

This invention also relates to the Combination Method, wherein the drug for which the major clearance mechanism in humans is CYP2D6 mediated oxidative biotransformation is a neurokinin-1 (NK-1) receptor antagonist containing a primary, secondary or tertiary alkylamine moiety.

This invention also relates to the Combination Method, wherein the drug for which the major clearance mechanism in humans is CYP2D6 mediated oxidative biotransformation is a tricyclic antidepressant containing a primary, secondary or tertiary alkylamine moiety (e.g., desipramine, imipramine or clomipramine).

A preferred embodiment of this invention relates to the Combination Method, wherein the drug for which the major clearance mechanism in humans is CYP2D6 mediated oxidative biotransformation, is (2S,3S)-2-phenyl-3-(2-methoxy-5-trifluoromethoxyphenyl)methylamino-piperidine or a pharmaceutically acceptable salt thereof.

A preferred embodiment of this invention relates to the Combination Method, wherein the drug for which the major clearance mechanism in humans is CYP2D6 mediated oxidative biotransformation, is sunipetron or a pharmaceutically acceptable salt thereof.

Sunipetron has the following structure

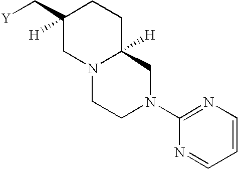

wherein Y is a group of the formula

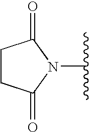

Another preferred embodiment of this invention relates to the Combination Method, wherein the drug for which the major clearance mechanism in humans is CYP2D6 mediated oxidative biotransformation is (1S,2S)-1-(4-hydroxyphenyl)-2-(4-hydroxy4-phenylpiperidin-1-yl)-1propanol or a pharmaceutically acceptable salt thereof.

Examples of other drugs for which the major clearance mechanism in humans is CYP2D6 mediated oxidative biotransformation are the following: mequitazine (J. Pharmacol. Exp. Ther., 284, 437442 (1998)); tamsulosin (Xenobiotica, 28, 909-22 (1998)); oxybutynin (Pharmacogen., 8, 449-51 (1998)); ritonavir (Clin. PK, 35, 275-291 (1998)); iloperidone (J. Pharmacol. Exp. Ther., 286, 1285-93 (1998)); ibogaine (Drug Metab. Dispos., 26, 764-8 (1998)); delavirdine (Drug Metab. Dispos., 26, 631-9 (1998)); tolteridine (Clin. Pharmcol. Ther., 63 529-39 (1998)); promethazine (Rinshoyakon, 29, 231-38 (1998)); pimozide, J. Pharmacol. Exp. Ther., 285, 428-37 (1998)); epinastine (Res. Comm. Md. Path. Pharmacol., 98, 273-92 (1997)); tramodol (Eur. J. Clin. Pharm., 53, 235-239 (1997)); procainamide (Pharmacogenetics, 7, 381-90 (1997)); methamphetamine (Drug Metab. Dispos., 25,1059-64 (1997)); tamoxifen (Cancer Res., 57, 3402-06 (1997)); nicergoline (Br. J. Pharm., 42, 707-11 (1996)); and fluoxetine (Clin. Pharmcol. Ther., 60, 512-21 (1996)). All of the foregoing references are incorporated herein by references in their entireties.

Examples of other drugs for which the major clearance mechanism in humans is CYP2D6 mediated oxidative biotransformation, all of which are referred to, along with their respective pathways of CPY2D6 mediated oxidative biotransformation (e.g., O-demethylation, hydroxylation, etc.), by M. F. Fromm et al. in Advanced Drug Delivery Reviews, 27, 171-199 (1997), are the following: alprenolol, amiflamine, amitriptyline, aprindine, brofaromine, buturalol, cinnarizine, clomipramine, codeine, debrisoquine, desipramine, desmethylcitalopram, dexfenfluramine, dextromethorphan, dihydrocodine, dolasetron, encainide, ethylmorphine, flecainide, flunarizine, fluvoxamine, guanoxan, haloperidol, hydrocodone, indoramin, imipramine, maprotiline, methoxyamphetamine, methoxyphenamine, methylenedioxymethamphetamine, metoprolol, mexiletine, mianserin, minaprine, procodeine, nortriptyline, N-propylajmaline, ondansetron, oxycodone, paroxetine, perhexiline, perphenazine, phenformine, promethazine, propafenone, propanolol, risperidone, sparteine, thioridazine, timolol, tomoxetine, tropisetron, venlafaxine and zuclopenthixol.

Other preferred embodiments of this invention relate to the Combination Method wherein the CYP2D6 inhibitor, or pharmaceutically acceptable salt thereof, that is employed in such method is quinidine or ajmalacine or a pharmaceutically acceptable salt of one of these compounds.

Other embodiments of this invention relate to the Combination Method, wherein the CYP2D6 inhibitor, or pharmaceutically acceptable salt thereof, that is employed in such method, is selected from the following compounds and their pharmaceutically acceptable salts: sertraline (J. Clin. Psychopharm. 18, 55-61 (1998)); venlafaxine (Br. J. Pharm., 43, 619-26 (1997)); dexmedetomidine (DMD, 25, 651-55 (1997)); tripennelamine, premethazine, hydroxyzine, (Drug Metab. Dispos., 26, 531-39 (1998)); halofrintane and chloroquine, (Br. J. Clin. Pharm., 45, 315-(1998)); and moclobemide (Psychopharm., 135, 22-26 (1998)).

A further embodiment of this invention relates to the Combination Method wherein the CYP2D6 inhibitor that is employed in such method is St. John's wort or an extract or constituent thereof.

This invention also relates to a pharmaceutical composition comprising:

(a) a therapeutically effective amount of a drug for which the major clearance mechanism in humans is CYP2D6 mediated oxidative biotransformation (also referred to throughout this document as a “Therapeutic Drug”), or a pharmaceutically acceptable salt thereof;

(b) an amount of a CYP2D6 inhibitor, or a pharmaceutically acceptable salt thereof, that is effective in treating the disorder or condition for which the Therapeutic Drug referred to in (a) is intended to treat; and

(c) a pharmaceutically acceptable carrier;

wherein said drug and said CYP2D6 inhibitor are not the same compound.

The above pharmaceutical composition is hereinafter referred to as the “Combination Pharmaceutical Composition”.

Preferred embodiments of this invention relate to Combination Pharmaceutical Compositions wherein the drug for which the major clearance mechanism in humans is CYP2D6 mediated oxidative biotransformation, or pharmaceutically acceptable salt thereof, that is contained in such pharmaceutical composition is (2S,3S)-2-phenyl-3-(2-methoxy-5-trifluoromethoxyphenyl)methylaminopiperidine or a pharmaceutically acceptable salt thereof.

Other preferred embodiments of this invention relate to Combination Pharmaceutical Compositions wherein the drug for which the major clearance mechanism in humans is CYP2D6 mediated oxidative biotransformation, or pharmaceutically acceptable salt thereof, that is contained in such pharmaceutical composition is (1S,2S)-1-(4-hydroxyphenyl)-2-(4-hydroxy-4-phenylpiperidin-1yl)-1propanol or a pharmaceutically acceptable salt thereof.

Other preferred embodiments of this invention relate to Combination Pharmaceutical Compositions wherein the drug for which the major clearance mechanism in humans is CYP2D6 mediated oxidative biotransformation, or pharmaceutically acceptable salt thereof, that is contained in such pharmaceutical composition is sunipetron or a pharmaceutically acceptable salt thereof.

Other embodiments of this invention relate to Combination Pharmaceutical Compositions wherein the drug for which the major clearance mechanism in humans is CYP2D6 mediated oxidative biotransformation, or pharmaceutically acceptable salt thereof, that is contained in such compositions is selected from the following compounds and their pharmaceutically acceptable salts: mequitazine (J. Pharmacol. Exp. Ther., 284, 437-442 (1998)); tamsulosin (Xenobiotica, 28, 909-22 (1998)); oxybutynin (Pharmacogen., 8, 449-51 (1998)); ritonavir (Clin. PK, 35, 275-291 (1998)); iloperidone (J. Pharmacol. Exp. Ther., 286, 1285-93 (1998)); ibogaine (Drug Metab. Dispos., 26, 764-8 (1998)); delavirdine (Drug Metab. Dispos., 26, 631-9 (1998)); tolteridine (Clin. Pharmcol. Ther., 63, 529-39 (1998)); promethazine (Rinshovakon, 29, 231-38 (1998)); pimozide, J. Pharmacol. Exp. Ther., 285, 428-37 (1998)); epinastine (Res. Comm. Md. Path. Pharmacol., 98, 273-92 (1997)); tramodol (Eur. J. Clin. Pharm., 53, 235-239 (1997)); procainamide (Pharmacogenetics, 7, 381-90 (1997)); methamphetamine (Drug Metab. Dispos., 25,1059-64 (1997)); tamoxifen (Cancer Res., 57, 3402-06 (1997)); nicergoline (Br. J. Pharm., 42, 707-11 (1996)); and fluoxetine (Clin. Pharmcol. Ther., 60, 512-21 (1996)). All of the foregoing references are incorporated herein by references in their entireties.

Other embodiments of this invention relate to Combination Pharmaceutical Compositions wherein the drug for which the major clearance mechanism in humans is CYP2D6 mediated oxidative biotransformation, or pharmaceutically acceptable salt thereof, that is contained in such compositions is selected from the following compounds and their pharmaceutically acceptable salts, all of which are referred to, along with their respective pathways of CYP2D6 mediated oxidative biotransformation (e.g., O-demethylation, hydroxylation, etc.), by M. F. Fromm et al. in Advanced Drug Delivery Reviews, 27, 171-199 (1997): alprenolol, amiflamine, amitriptyline, aprindine, brofaromine, buturalol, cinnarizine, clomipramine, codeine, debrisoquine, desipramine, desmethylcitalopram, dexfenfluramine, dextromethorphan, dihydrocodine, dolasetron, encainide, ethylmorphine, flecainide, flunarizine, fluvoxamine, guanoxan, haloperidol, hydrocodone, indoramin, imipramine, maprotiline, methoxyamphetamine, methoxyphenamine, methylenedioxymethamphetamine, metoprolol, mexiletine, mianserin, minaprine, procodeine, nortriptyline, N-propylajmaline, ondansetron, oxycodone, paroxetine, perhexiline, perphenazine, phenformine, promethazine, propafenone, propanolol, risperidone, sparteine, thioridazine, timolol, tomoxetine, tropisetron, venlafaxine and zuclopenthixol.

Other embodiments of this invention relate to Combination Pharmaceutical Compositions wherein the CYP2D6 inhibitor, or pharmaceutically acceptable salt thereof, that is contained in such composition is selected from the following compounds and their pharmaceutically acceptable salts: sertraline (J. Clin. Psychopharm., 18, 55-61 (1998)); venlafaxine (Br. J. Pharm., 43, 619-26 (1997)); dexmedetomidine (DMD, 25, 651-55 (1997)); tripennelamine, premethazine, hydroxyzine, (Drug Metab. Dispos., 26, 531-39 (1998)); halofrintane and chloroquine, (Br. J. Clin. Pharm., 45, 315-(1998)); and moclobemide (Psychopharm., 135, 22-26 (1998)).

A further embodiment of this invention relates to the Combination Method wherein the CYP2D6 inhibitor that is employed in such method is St. John's wort or an extract or constituent thereof.

This invention also relates to a Combination Pharmaceutical Composition, wherein the drug for which the major clearance mechanism in humans is CYP2D6 mediated oxidative biotransformation is a selective serotonin reuptake inhibitor containing a primary, secondary or tertiary alkylamine moiety (e.g., sertraline or fluoxetine).

This invention also relates to a Combination Pharmaceutical Composition, wherein the drug for which the major clearance mechanism in humans is CYP2D6 mediated oxidative biotransformation is an NMDA (N-methyl-D-aspartate) receptor antagonist containing a primary, secondary or tertiary alkylamine moiety.

This invention also relates to a Combination Pharmaceutical Composition, wherein the drug for which the major clearance mechanism in humans is CYP2D6 mediated oxidative biotransformation is an a neurokinin-1(NK-1) receptor antagonist containing a primary, secondary or tertiary alkylamine moiety.

This invention also relates to a Combination Pharmaceutical Composition, wherein the drug for which the major clearance mechanism in humans is CYP2D6 mediated oxidative biotransformation is a tricyclic antidepressant containing a primary, secondary or tertiary alkylamine moiety (e.g., desipramine, imipramine or clomipramine).

The term “treatment”, as used herein, refers to reversing, alleviating, inhibiting the progress of, or preventing the disorder or condition to which such term applies, or one or more symptoms of such condition or disorder. The term “treatment”, as used herein, refers to the act of treating, as “treating” is defined immediately above.

The term “CYP2D6 mediated oxidative transformation”, as used herein, refers to the CYP2D6 catalyzed oxidation reactions (e.g., benzylic, aromatic or aliphatic hydroxylation, O-dealkylation, N-dealkylation, sidechain, sulfoxidation) through which metabolism of CPY2D6 substrate drugs proceeds.

### DETAILED DESCRIPTION OF THE INVENTION

This invention relates both to Combination Methods, as defined above, in which the Therapeutic Drug, or pharmaceutically acceptable salt thereof, and the CYP2D6 inhibitor, or pharmaceutically acceptable salt thereof, are administered together, as part of the same pharmaceutical composition, and to Combination Methods in which these two active agents are administered separately as part of an appropriate dose regimen designed to obtain the benefits of the combination therapy.

The appropriate dose regimen, the amount of each dose administered, and specific intervals between doses of each active agent will depend on the patient being treated, and the source and severity of the condition. Generally, in carrying out the methods of this invention, the Therapeutic Drug will be administered in an amount ranging from one order of magnitude less than the amount that is known to be efficacious and therapeutically acceptable for use of the Therapeutic Drug alone (i.e., as a single active agent) to the amount that is known to be efficacious and therapeutically acceptable for use of the Therapeutic Drug alone. For example, (2S,3S)-2-phenyl-3-(2-methoxy-5-trifluoromethoxyphenyl)methylaminopiperidine will generally be administered to an average weight (approximately 70 kg) adult human in an amount ranging from about 5 to about 1500 mg per day, in single or divided doses, preferably from about 0.07 to about 21 mg/kg. (1S,2S)-1-(4-hydroxyphenyl)-2-(4-hydroxy-4-phenylpiperidin-1yl)-1or a pharmaceutically acceptable salt thereof will generally be administered to an average weight adult human in an amount ranging from about 0.02 to about 250 mg per day, in single or divided doses, preferably from about 0.15 to about 250 mg per day. Sunipetron will generally be administered to an average weight adult human in an amount ranging from about 2 to about 200 mg per day, in single or divided doses. Variations may nevertheless occur depending upon the physical condition of the patient being treated and his or her individual response to said medicament, as well as on the type of pharmaceutical formulation chosen and the time period and interval at which such administration is carried out. In some instances, dosage levels below the lower limit of the aforesaid range may be more than adequate, while in other cases still larger doses may be employed without causing any harmful side effect, provided that such larger doses are first divided into several small doses for administration throughout the day.

The Therapeutic Drugs, e.g., (7S,9S)-2-(2-pyrimidyl)-7-(succinamidomethyl)-prehydro-1 H-pyrido-[1,2-a]pyrazine) (“sunipetron”), (2S,3S)-2-phenyl-3-(2-methoxyphenyl)-methylaminopiperidine, (1S,2S)-1-(4-hydroxyphenyl)-2-(4-hydroxy-4-phenylpiperidin-1yl)-1propanol, (2S,3S)-2-phenyl-3-(2-methoxy-5-trifluoromethoxyphenyl)methylaminopiperidine, and the CYP2D6 inhibitor compounds and their pharmaceutically acceptable salts (both the Therapeutic Drugs and the CYP2D6 inhibitors, as well as their pharmaceutically acceptable salts, hereinafter, also referred to individually or collectively, as “active agents”) can each be administered separately or can be administered together, each or both in combination with pharmaceutically acceptable carriers or diluents in single or multiple doses. More particularly, such agents can be administered in a wide variety of different dosage forms, i.e., they may be combined with various pharmaceutically acceptable inert carriers in the form of tablets, capsules, lozenges, troches, hard candies, powders, sprays, creams, salves, suppositories, jellies, gels, pastes, lotions, ointments, aqueous suspensions, injectable solutions, elixirs, syrups, and the like. Such carriers include solid diluents or fillers, sterile aqueous media and various non-toxic organic solvents, etc. Moreover, oral pharmaceutical compositions can be suitably sweetened and/or flavored. In general, each or both of the foregoing active agents is present in such dosage forms at concentration levels ranging from about 5.0% to about 70% by weight.

For oral administration, tablets containing various excipients such as microcrystalline cellulose, sodium citrate, calcium carbonate, dicalcium phosphate and glycine may be employed along with various disintegrants such as starch (and preferably corn, potato or tapioca starch), alginic acid and certain complex silicates, together with granulation binders like polyvinylpyrrolidone, sucrose, gelatin and acacia. Additionally, lubricating agents such as magnesium stearate, sodium lauryl sulfate and talc are often very useful for tabletting purposes. Solid compositions of a similar type may also be employed as fillers in gelatin capsules; preferred materials in this connection also include lactose or milk sugar as well as high molecular weight polyethylene glycols. When aqueous suspensions and/or elixirs are desired for oral administration, the active ingredient may be combined with various sweetening or flavoring agents, coloring matter or dyes, and, if so desired, emulsifying and/or suspending agents as well, together with such diluents as water, ethanol, propylene glycol, glycerin and various like combinations thereof.

For parenteral administration, solutions of either or both of the active agents, or pharmaceutically acceptable salts thereof, employed in the methods of this invention in either sesame or peanut oil or in aqueous propylene glycol may be used. The aqueous solutions should be suitably buffered (preferably pH greater than 8) if necessary and the liquid diluent first rendered isotonic. These aqueous solutions are suitable for intravenous injection purposes. The oily solutions are suitable for intraarticular, intramuscular and subcutaneous injection purposes. The preparation of all these solutions under sterile conditions is readily accomplished by standard pharmaceutical techniques well known to those skilled in the art.

Additionally, it is also possible to administer either or both the active agents, or pharmaceutically acceptable salts thereof, employed in the methods of this invention topically when treating inflammatory conditions of the skin, and this may be done by way of creams, jellies, gels, pastes, patches, ointments and the like, in accordance with standard pharmaceutical practice.

Whether a person is a “poor metabolizer” or an “extensive metabolizer” can be determined by measuring the concentrations of the drug dextromethorphan and its metabolite dextrorphan in the person's blood, urine or saliva after passage of a period of time following administration of the drug. A dextromethorphan/dextrorphan ratio of less than 0.3 defines an extensive metabolizer, while the same ratio greater than or equal to 0.3 defines a poor metabolizer. Suitable periods of time to wait after administration of the drug for this type of phenotyping are: from about 4 to 8 hours for urine measurements, 2 to 8 hours for plasma measurements and three to 8 hours for saliva measurements. Such a method is described by Schmidt et al., Clin. Pharmacol. Ther., 38, 618, 1985.

The following protocol can be used to determine the impact that coadministration of a CYP2D6 inhibitor with a Therapeutic Drug, as defined above, would have on the pharmacokinetics of the Therapeutic Drug.

Method:

1.Subjects that are predetermined to be extensive metabolizers (EMs; those individuals with functional CYP2D6 activity) are administered an oral dose of a compound being tested as a CYP2D6 inhibitor.

2. Concomitantly, or at some predetermined time period after the dose of the CYP2D6 inhibitor, these subjects are administered a dose of a drug known to be primarily cleared via CYP2D6 mediated metabolism.

3. At times of 0 hour (predose) and at predetermined time points after administration of the CYP2D6 cleared compound, several blood samples are taken from each subject. An example of sampling times would be 0.5, 1, 2, 3, 4, 6, 8, 12, 18, 24, 36, 48, and 72 hours.

4. The blood (or plasma or serum) is analyzed for the CYP2D6 cleared compound using a specific bioanalytical method (such as HPLC with UV or MS detection).

5. The blood concentrations of the CYP2D6 cleared compound are plotted vs time, and pharmacokinetics are calculated from these data. The pharmacokinetic parameters to be measured are the area under the concentration vs. time curve (AUC), maximum concentration (Cₘₐₓ), time of maximum concentration (Tₘₐₓ), clearance (CL), and half-life (t_{1/2}).

6. A second leg of the experiment involves dosing the same subjects with the CYP2D6 cleared compound in the absence of the CYP2D6 inhibitor. Steps 3-5 are repeated. (The order of the two legs of this study is not important, as long as a suitable washout period is applied.)

7. The concentration vs. time plots and the pharmacokinetic parameters from the two legs of the study are compared and the effect of the CYP2D6 inhibitor assessed by this comparison.

1. A method of administering a drug for which the major clearance mechanism in humans is CYP2D6 mediated oxidative biotransformation, or a pharmaceutically acceptable salt thereof, in combination with a CYP2D6 inhibitor, or a pharmaceutically acceptable salt thereof, to a human in need of the intended pharmaceutical activity of such drug, wherein said drug and said CYP2D6 inhibitor are not the same compound.

2. A method according to claim 1 wherein the drug for which the major clearance mechanism in humans is CYP2D6 mediated oxidative biotransformation is a selective serotonin reuptake inhibitor containing a primary, secondary or tertiary alkylamine moiety or a pharmaceutically acceptable salt thereof.

3. A method according to claim 1 wherein the drug for which the major clearance mechanism in humans is CYP2D6 mediated oxidative biotransformation is an NMDA receptor antagonist containing a primary, secondary or tertiary alkylamine moiety or a pharmaceutically acceptable salt thereof.

4. A method according to claim 1 wherein the drug for which the major clearance mechanism in humans is CYP2D6 mediated oxidative biotransformation is a neurokinin-1 (NK-1) receptor antagonist containing a primary, secondary or tertiary alkylamine moiety or a pharmaceutically acceptable salt thereof.

5. A method according to claim 1 wherein the drug for which the major clearance mechanism in humans is CYP2D6 mediated oxidative biotransformation is a tricyclic antidepressant containing a primary, secondary or tertiary alkylamine moiety or a pharmaceutically acceptable salt thereof.

6. A method according to claim 1, wherein the drug for which the major clearance mechanism in humans is CYP2D6 mediated oxidative biotransformation is (2S,3S)-2-phenyl-3-(2-methoxy-5-trifluoromethoxyphenyl)methylamino-piperidine, or a pharmaceutically acceptable salt thereof.

7. A method according to claim 1, wherein the drug for which the major clearance mechanism in humans is CYP2D6 mediated oxidative biotransformation is (1S, 2S)-l -(4-hydroxyphenyl)-2-(4-hydroxy4-phenylpiperid in-1yl)-1-propanol or a pharmaceutically acceptable salt thereof.

8. A method according to claim 1, wherein the drug for which the major clearance mechanism in humans is CYP2D6 mediated oxidative biotransformation is sunipetron or a pharmaceutically acceptable salt thereof.

9. A method according to claim 1, wherein the drug for which the major clearance mechanism in humans is CYP2D6 mediated oxidative biotransformation is selected from the group consisting of mequitazine, tamsulosin, oxybutynin, ritonavir, iloperidone, ibogaine, delavirdine, tolteridine, promethazine, pimozide, epinastine, tramodol, procainamide, methamphetamine, tamoxifen, nicergoline, fluoxetine, and pharmaceutically acceptable salts thereof.

10. A method according to claim 1, wherein the drug for which the major clearance mechanism in humans is CYP2D6 mediated oxidative biotransformation is selected from the group consisting of alprenolol, amiflamine, amitriptyline, aprindine, brofaromine, buturalol, cinnarizine, clomipramine, codeine, debrisoquine, desipramine, desmethylcitalopram, dexfenfluramine, dextromethorphan, dihydrocodine, dolasetron, encainide, ethylmorphine, flecainide, flunarizine, fluvoxamine, guanoxan, haloperidol, hydrocodone, indoramin, imipramine, maprotiline, methoxyamphetamine, methoxyphenamine, methylenedioxymethamphetamine, metoprolol, mexiletine, mianserin, minaprine, procodeine, nortriptyline, N-propylajmaline, ondansetron, oxycodone, paroxetine, perhexiline, perphenazine, phenformine, promethazine, propafenone, propanolol, risperidone, sparteine, thioridazine, timolol, tomoxetine, tropisetron, venlafaxine, zuclopenthixol, and pharmaceutically acceptable salts thereof.

11. A method according to claim 1, wherein the CYP2D6 inhibitor is quinidine, ajmalacine or pharmaceutically acceptable salts thereof.

12. A method according to claim 1, wherein the CYP2D6 inhibitor is selected from the group consisting of sertraline, venlafaxine, dexmedetomidine, tripennelamine, premethazine, hydroxyzine, halofrintane, chloroquine, moclobemide, and pharmaceutically acceptable salts thereof.

13. A method according to claim 1, wherein the CYP2D6 inhibitor is St. John's wort, or an extract or constituent thereof.

14. A pharmaceutical composition comprising:
(a) a therapeutically effective amount of a drug for which the major clearance mechanism in humans is CYP2D6 mediated oxidative biotransformation, or a pharmaceutically acceptable salt thereof;
(b) an amount of a CYP2D6 inhibitor, or a pharmaceutically acceptable salt thereof, that is effective in treating the disorder or condition for which the drug referred to in “a” is intended to treat; and
(c) a pharmaceutically acceptable carrier;
wherein said drug and said CYP2D6 inhibitor are not the same compound.

15. A pharmaceutical composition according to claim 14, wherein the drug for which the major clearance mechanism in humans is CYP2D6 mediated oxidative biotransformation is (2S,3S)-2-phenyl-3-(2-methoxy-5-trifluoromethoxy-phenyl)methyl-aminopiperidine or a pharmaceutically acceptable salt thereof.

16. A pharmaceutical composition according to claim 14, wherein the drug for which the major clearance mechanism in humans is CYP2D6 mediated oxidative biotransformation is sunipetron or a pharmaceutically acceptable salt thereof.

17. A pharmaceutical composition according to claim 14, wherein the drug for which the major clearance mechanism in humans is CYP2D6 mediated oxidative biotransformation is (1S,2S)-1-(4-hydroxyphenyl)-2-(4-hydroxy-4-phenylpiperidin-1-yl)-1-propanol or a pharmaceutically acceptable salt thereof.

18. A pharmaceutical composition according to claim 14, wherein the drug for which the major clearance mechanism in humans is CYP2D6 mediated oxidative biotransformation is selected from the group consisting of mequitazine, tamsulosin, oxybutynin, ritonavir, iloperidone, ibogaine, delavirdine, tolteridine, promethazine, pimozide, epinastine, tramodol, procainamide, methamphetamine, tamoxifen, nicergoline, fluoxetine, and pharmaceutically acceptable salts thereof.

19. A pharmaceutical composition according to claim 14, wherein the drug for which the major clearance mechanism in humans is CYP2D6 mediated oxidative biotransformation is selected from the group consisting of alprenolol, amiflamine, amitriptyline, aprindine, brofaromine, buturalol, cinnarizine, clomipramine, codeine, debrisoquine, desipramine, desmethylcitalopram, dexfenfluramine, dextromethorphan, dihydrocodine, dolasetron, encainide, ethylmorphine, flecainide, flunarizine, fluvoxamine, guanoxan, haloperidol, hydrocodone, indoramin, imipramine, maprotiline, methoxyamphetamine, methoxyphenamine, methylenedioxymethamphetamine, metoprolol, mexiletine, mianserin, minaprine, procodeine, nortriptyline, N-propylajmaline, ondansetron, oxycodone, paroxetine, perhexiline, perphenazine, phenformine, promethazine, propafenone, propanolol, risperidone, sparteine, thioridazine, timolol, tomoxetine, tropisetron, venlafaxine, zuclopenthixol and pharmaceutically acceptable salts thereof.

20. A pharmaceutical composition according to claim 14, wherein the CYP2D6 inhibitor is quinidine, ajmalacine or pharmaceutically acceptable salts thereof.

21. A pharmaceutical composition according to claim 14, wherein the CYP2D6 inhibitor is selected from the group consisting of sertraline, venlafaxine, dexmedetomidine, tripennelamine, premethazine, hydroxyzine, halofrintane, chloroquine, moclobemide, and pharmaceutically acceptable salts thereof.

22. A pharmaceutical composition according to claim 14, wherein the CYP2D6 inhibitor is St. John's wort, or an extract or constituent thereof.
